(19) United States
(12) Patent Application Publication
(10) Pub. No.: US 2004/0172680 A1
(43) Pub. Date: Sep. 2, 2004

(54) Process for modifying plants

(76) Inventors: Mark Harker, Shambrook (GB); Susan Amanda Hellyer, Shambrook (GB); Niklas Holmberg, Shambrook (GB); Dick Safford, Shambrook (GB)

Correspondence Address: UNILEVER, PATENT DEPARTMENT, 45 RIVER ROAD, EDGEWATER, NJ, 07020, US

(21) Appl. No.: 10/257,344

(22) PCT Filed: Apr. 4, 2001
(86) PCT No.: PCT/EP01/03808

(30) Foreign Application Priority Data
Apr. 14, 2000 (EP) 00303193.7

(51) Int. Cl. A01H 1/00; C12N 15/82

(52) U.S. Cl. 800/281

(57) ABSTRACT

The use of a gene expressing a SMT1 to increase the level of sterols or decrease the amount of cholesterol in the seeds of plants.

### FIELD OF THE INVENTION

The invention relates to a process for the modification of plants, more specifically a process for increasing the isoprenoid level in plants.

### BACKGROUND OF THE INVENTION

Many approaches have been suggested for modifying the isoprenoid production in plants.

Whereas only a few sterols exist in animals, with cholesterol being by far the major one, in plants a wide range of sterols are found. Structural variations between these arise from different substitutions in the side chain and the number and position of double bonds in the tetracyclic skeleton. Plant sterols can be grouped by the presence or absence of one or more functionalities. For example they can be divided into three groups based on ethylation levels at C4 as follows: 4-desmethylsterols or end product sterols, 4α-monomethylsterols and 4,4-di-ethylsterols. Naturally occurring 4-desmethylsterols include sitosterol, stigmasterol, brassicasterol, campesterol, avenasterol and isofucosterol. In most higher plants, sterols with a free 3β-hydroxyl group (free sterols) are the major end products. However, sterols also occur as conjugates, for example, where the 3-hydroxy group is esterified by a fatty acid chain or phenolic acid to give a steryl ester. For the purpose of this description, the term sterol refers both to free sterols and conjugated sterols. However in this specification references to levels, amounts or percentages of sterol refer to the total weight sterol groups whereby the weight of the conjugating groups such as fatty acid or phenolic acid is excluded.

To date most studies aimed at manipulating sterols in plants had the purpose of increasing resistance to pests or to fungicides.

WO 98/45457 describes the modulation of phytosterol compositions to confer resistance to insects, nematodes, fungi and/or environmental stresses, and/or to improve the nutritional value of plants by using a double stranded DNA molecule comprising a promoter, a DNA sequence encoding a first enzyme which binds a first sterol and produces a second sterol and a 3′ non-translated region which causes polyadenylation at the 3′ end of the RNA. Preferably the enzyme is selected from the group consisting of S-adenosyl-L-methionine-Δ²⁴⁽²⁵⁾-sterol methyl transferase, a C-4 demethylase, a cycloeucalenol to obtusifoliol-isomerase, a 14-α-demethylase, a Δ⁸ to Δ⁷-isomerase, a Δ⁷-C-5-desaturase and a 24,25-reductase.

U.S. Pat. No. 5,306,862 describes a method of increasing sterol accumulation in a plant by increasing the copy number of a gene encoding a polypeptide having HMG-CoA reductase activity to increase the resistance of plants to pests. Similarly U.S. Pat. No. 5,349,126 discloses a process to increase the squalene and sterol accumulation in transgenic plants by increasing the amount of a gene encoding a polypeptide having HMG-CoA reductase activity to increase the pest resistance of transgenic plants.

WO 97/48793 discloses a C-14 sterol reductase polypeptide for the genetic manipulation of a plant sterol biosynthetic pathway.

WO 96/09393 discloses a DNA sequence encoding squalene synthetase.

WO 97/34003 discloses a process of raising squalene levels in plants by introduction into a genome of a plant a DNA to suppress expression of squalene epoxidase.

WO 93/16187 discloses new plants containing in its genome one or more genes involved in the early stages of phytosterol biosynthesis, preferably the genes encode mevalonate kinase.

U.S. Pat. No.5,589,619 discloses accumulation of squalene in plants by introducing a HMG-CoA reductase gene to increase production of sterol and resistance to pests. Example 10 discloses increased squalene levels in the seeds of these plants.

WO 00/08190 discloses a DNA sequence encoding a sterol methyltransferase isolated from Zea mays.

In plants, 24-methylene cycloartanol production from cycloartenol via SMT1 is one of the steps in isoprenoid biosynthesis.

Bouvier-Navé et al in Eur. J. Biochem. 256, 88-96 (1988) describes two families of sterol methyl transferases (SMTs), The first (SMT1) applying to cycloartenol and the second (SMT2) to 24-methylene lophenol.

Schaller et al in Plant Physiology (1998) 118: 461-169 describes the over-expression of SMT2 from Arabidopsis in tobacco resulting in a change in the ratio of 24-methyl cholesterol to sitosterol in the tobacco leaf.

The present invention aims to modify sterol levels in the seeds of plants, whereby this modification can either involve an increase of the level of (beneficial) sterols or a decrease of the level of (less-desired) cholesterol.

It has been found that genes expressing specific sterol methyl transferases can advantageously be used to modify the nutritional value of plants especially in the seeds thereof. Surprisingly it has been found that the use of SMT1 leads to the enhancement of nutritionally beneficial sterols and/or the reduction of cholesterol in the seeds of said plants. Surprisingly the reduction of cholesterol is also observed in other plant tissue.

### STATEMENT OF THE INVENTION

Accordingly the invention relates to the use of a gene expressing a SMT1 to increase the level of sterols in the seeds of plants and/or decrease the level of cholesterol in plant tissue.

In another aspect the invention relates to a method to increase the level of sterols in the seeds of plants and/or decrease the level of cholesterol in plant tissue by increasing the expression of SMT1.

### DETAILED DESCRIPTION OF THE INVENTION

In higher plants, isoprenoids are a large family of compounds with diverse roles. They include sterols, the plant hormones gibberellins and abscisic acid, components of photosynthetic pigments, phytoalexins and a variety of other specialised terpenoids.

Sterols, especially 4-desmethylsterols or precursors therefore such as cycloartenol are of interest because 4-desmethylsterols contribute to the nutritional quality, flavour and colour of fruits and vegetable oils. Of particular interest are isoprenoid compounds of nutritional benefit such as fat-soluble sterols. These may be efficacious in reducing coronary heart disease, for example, some phytosterols have been shown to lower serum cholesterol levels when increased in the diet and vitamin E reduces atheroscelerotic plaques via decreased oxidation of LDL.

Expression of such compounds in plant seeds, in particular in oilseeds, is commercially advantageous as generally the harvesting of such ingredients from seeds is very convenient and, in some instances, it may be possible to extract the oil in combination with the sterols from the seed, leading to an oil containing elevated levels of sterol without or with the reduced need for separate addition of sterols.

Preferred sterols are 4-desmethylsterols, most preferred betasitosterol, sitostanol, stigmasterol, brassicasterol, campestanol, isofucosterol and campesterol. Also preferably, at least part of the sterols, for example at least 50 wt % based on the total of the sterols in the seed are esters of sterols with C10-24 fatty acids. In a very preferred embodiment the sterols comprise C10-24 esters of 4-desmethylsterols.

Cholesterol is a less desired component of food products because consumers have a desire to reduce their cholesterol consumption. It is believed that reduced serum cholesterol levels lead to a reduced risk of cardiovascular disease. Therefore in one embodiment the invention relates to the reduction of the cholesterol level in plant tissue, especially the seeds of plants.

As discussed above, several approaches have been suggested to alter the levels of isoprenoids and/or cholesterol in plants. It has now been found that for the enhancement of isoprenoid levels in seeds a preferred route is to use a SMT1 gene. The use of such genes is especially advantageous to enhance the levels of 4-desmethylsterols,even more preferred the level of stigmasterol, sitosterol, brassicasterol, isofucosterol and campesterol in seeds. Also, the use of such genes is especially advantageous to enhance the levels of isoprenoids in oilseeds containing more than 10 wt % based on dry weight of triacylglycerols.

Suitably the SMT1 gene can be naturally present in the plant. In accordance to the invention the circumstances are then altered such that increased expression of SMT1, preferably in the seed region of the plant will take place. Possible ways to do this may be to upregulate facilitating molecules e.g. such as transcription factors. Alternatively a specific promoter can be inserted into the plant genome to ensure that the SMT1 gene is upregulated. Alternatively the copy number of the “homologous” SMT1 gene may be increased to increase the expression thereof.

Alternatively the SMT1 gene can be a heterologous gene, for example derived from other plant or microbial sources. For example the SMT1 gene may be derived from Arabidopsis, tobacco or yeast.

The invention also provides a method of transforming a plant by

a) transforming a plant cell with a recombinant DNA construct comprising a DNA segment encoding a polypeptide with SMT1 activity and a promoter for driving the expression of said polypeptide in said plant cell to form a transformed plant cell.

b) regenerating the transformed plant cell into the transgenic plant.

c) selecting transgenic plants that have enhanced levels of sterols and/or decreased levels of cholesterol in the seeds compared to wild type strains of the same plant.

DNA segments encoding SMT1 for use according to the present invention may suitably be obtained from animals, microbial sources or plants, Alternatively, equivalent genes could be isolated from gene libraries, for example by hybridisation techniques with DNA probes.

The gene sequences of interest will be operably linked (that is, positioned to ensure the functioning of) to one or more suitable promoters which allow the DNA to be transcribed. Suitable promoters, which may be homologous or heterologous to the gene, useful for expression in plants are well known in art, as described, for example, in Weising et al, (1988), Ann. Rev. Genetics, 22, 421-477). Promoters for use according to the invention may be inducible, constitutive or tissue-specific or have various combinations of such characteristics. Useful promoters include, but are not limited to constitutive promoters such as carnation etched ring virus (CERV), cauliflower mosaic virus (CaMV) 35S promoter, or more particularly the double enhanced cauliflower mosaic virus promoter, comprising two CaMV 35S promoters in tandem (referred to as a “Double 35S”promoter).

It may be desirable to use a tissue-specific or developmentally regulated promoter instead of a constitutive promoter in certain circumstances. A tissue-specific promoter allows for overexpression in certain tissues without affecting expression in other tissues. By way of illustration, a preferred promoter used in overexpression of enzymes in seed tissue is an ACP promoter as described in W092/18634.

The promoter and termination regulatory regions will be functional in the host plant cell and may be heterologous (that is, not naturally occurring) or homologous (derived from the plant host species) to the plant cell and the gene. Suitable promoters which may be used are described above.

The termination regulatory region may be derived from the 3′ region of the gene from which the promoter was obtained or from another gene. Suitable termination regions which may be used are well known in the art and include Agrobacterium tumefaciens nopaline synthase terminator (Tnos), Agrobacterium tumefaciens mannopine synthase terminator (Tmas) and the CaMV 35S terminator (T35S). Particularly preferred termination regions for use according to the invention include the pea ribulose bisphosphate carboxylase small subunit termination region (TrbcS) or the Tnos termination region.

Such gene constructs may suitably be screened for activity by transformation into a host plant via Agrobacterium and screening for increased isoprenoid levels.

Suitably, the nucleotide sequences for the genes may be extracted from the Genbank nucleotide database and searched for restriction enzymes that do not cut. These restriction sites may be added to the genes by conventional methods such as incorporating these sites in PCR primers or by sub-cloning.

Preferably the DNA construct according to the invention is comprised within a vector, most suitably an expression vector adapted for expression in an appropriate host (plant) cell. It will be appreciated that any vector which is capable of producing a plant comprising the introduced DNA sequence will be sufficient.

Suitable vectors are well known to those skilled in the art and are described in general technical references such as Pouwels et al, Cloning Vectors. A laboratory manual, Elsevier, Amsterdam (1986). Particularly suitable vectors include the Ti plasmid vectors.

Transformation techniques for introducing the DNA constructs according to the invention into host cells are well known in the art and include such methods as micro-injection, using polyethylene glycol, electroporation, or high velocity ballistic penetration. A preferred method for use according to the present invention relies on agrobacterium—mediated transformation.

After transformation of the plant cells or plant, those plant cells or plants into which the desired DNA has been incorporated may be selected by such methods as antibiotic resistance, herbicide resistance, tolerance to amino-acid analogues or using phenotypic markers.

Various assays may be used to determine whether the plant cell shows an increase in gene expression, for example, Northern blotting or quantitative reverse transcriptase PCR (RT-PCR). Whole transgenic plants may be regenerated from the transformed cell by conventional methods. Such transgenic plants having improved isoprenoid levels may be propagated and self-pollinated to produce homozygous lines. Such plants produce seeds containing the genes for the introduced trait and can be grown to produce plants that will produce the selected phenotype.

Preferably the level of sterols, especially the total level of campesterol, stigmasterol, sitosterol and isofucosterol in the seeds of the plants is at least 5wt % more than the level in corresponding plants without the SMT1 gene, more preferred more than 10% more, especially preferred more than 25% more, most preferred more than 30% more, for example about 35% more. Especially preferably the total level of campesterol, stigmasterol, sitosterol and isofucosterol in the seeds of the plants is at least 0.400% on dry weight.

The invention also provides seeds obtained from oil plants with a upregulated SMT1 gene, especially preferred oilseeds are tobacco seeds, canola seeds, rapeseed, sunflower seeds and soybean seeds. Also provided is a method to extract oil, whereby the oil is extracted from these seeds. Any suitable method can be used for such extraction.

The invention will now be further illustrated in the following examples.

### EXAMPLE 1

### Cloning the SMT1 Gene from Nicotiana Tabacum (Ntsmt1)

E. coli strain DH5 α (Gibco BRL) was used as the host strain in all cloning procedures. Pre-digested vector pGEM-T Easy containing poly-T overhangs was obtained from Promega and used according to the suppliers' recommendations. Bacteria were cultivated in LB medium (10 g/l tryptone, 5g/l yeast extract, 5 g/l NaCl) supplemented with ampicillin (100 μg/ml) on a rotary shaker (210 rpm) at 37° C.

Restriction endonucleases, T4 DNA ligase, molecular markers (X, XIV and XVII) and Taq DNA polymerase were purchased from Roche. Pfu DNA polymerase and reverse transcriptase (Superscript I) were obtained from Stratagene and Gibco BRL, respectively. The enzymes were used according to the suppliers' recommendations. All chemicals and reagents used were of analytical grade and commercially available. All oligonucleotide primers (given in 5′ to 3′ direction) are listed in Table 1A.

TABLE 1A

| | |
|---|---|
| Primer | Sequence |

| | |
|---|---|
| RoRidT | AAG GAT CCG TCG ACA TCG ATA ATA CGA CTC |
| | ACTATA GGG ATT TTT TTT TTT TTT TTT TTT |
| Ntsmt13 | CAA CGC CCT CCA AAA ACT GTA |
| Ntsmt15 | TGG GAA AGA AAT GTC AAA A |
| pUC/M13 | TTT CCC AGT CAC GAC GTT GT |
| Forward | |
| pUC/M13 | GTA AAA CGA CGG CCA GT |
| Reverse | |
| ACP5 | CCA TCG ATC TGA TTG GTA AGA TAT GG |
| Clsmt5* | GGA AGG ACA TGT CAA AAC AAG GG |
| Clsmt3# | CGG AAT TCC ATA TTA CTG AGA |
| CERV1S | GTC TGT CTA AAG TAA AGT AGA TGC G |
| NosAs | CCG GCA ACA GGA TTC AAT CTT |
| Seqsmt3 | AGC TAC TGA GCG AGA AGT G |
| Seqsmt5 | GCA CCC AGG TGG AAA GG |

The primers hold the following restriction enzyme sites (underlined): *AflIII and ^{#}EcoRI.

Two week old seedlings of tobacco (SR 1, Petit Havanna) were ground up in a mortar and the total RNA was isolated using the Purescript RNA isolation kit from Flowgene.

Total RNA (5 μg) was mixed with primer RoRidT (10 pmol) in 11.34 μl DEPC- treated mQ water. The mixture was incubated at 68° C. for 4 minutes and thereafter placed on wet ice for 2 minutes. First strand buffer (1×), DTT (0.1 μmol), RNAsin (22 U), dNTP (20 nmol) and Superscript (200 U) was added to give a final volume of 20 μl. The mix was incubated at 37° C. for 60 minutes.

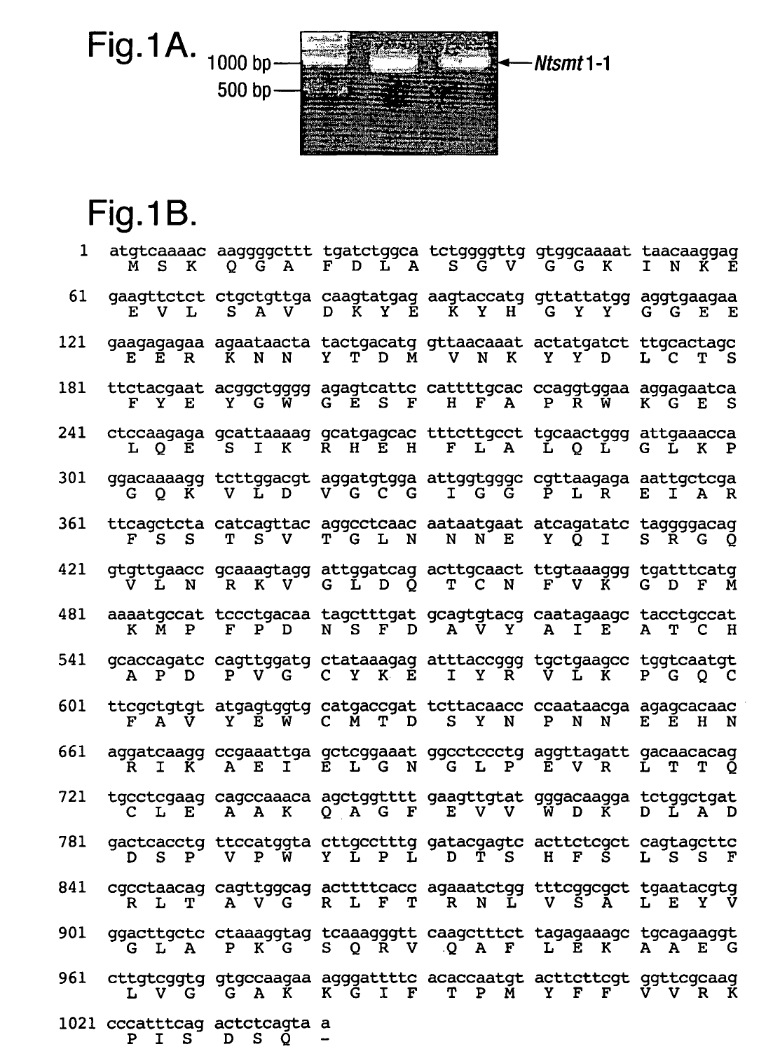

The Ntsmtl-1 cDNA was amplified by gene specific primers (Ntsmt13 and Ntsmt15) using 35 thermal cycles (30 s. 94° C., 30 s. 53° C., 90 s. 72° C.) and a mix of Taq and Pfu (10%) DNA polymerase to enhance the fidelity. The amplification products were separated on an agarose gel (1.2%) and the fragment corresponding to the full length Ntsmt1-1 cDNA (FIG. 1A) was excised and ligated into pGEM-T Easy. Clones containing the amplification product were selected based on blue-white screening using plates containing ampicillin (100 μg/ml), X-gal (80 μg/mL) and IPTG (0.5 mM).

Plasmid DNA was isolated by the boiling preparation method when screening potential positive transformants. Two ml over-night culture was pelleted (1 min., 15,000 rpm). The pellet was resuspended in 300 μl chilled STET buffer (8% Sucrose, 0.5% Triton X-100, 50 mM EDTA, 100 mM Tris-HCl, pH 8.0) and 30 μl lysozyme solution (10 mg/ml) was added. The cell suspension was boiled for 45 s. and subsequently centrifuged (15 min., 15000 rpm, 4° C.). The supernatant was mixed with 300 μl isopropanol, incubated at room temperature (15 min.) and centrifuged (15 min., 15000 rpm, 4° C.). The supernatant was removed and the pellet was air dried and finally dissolved in 50 μl TE-Buffer. The Promega mini prep kit was used to obtain plasmid DNA for sequencing.

The PCR amplified Ntsmtl-l gene was sequenced using an automatic Perkin Elmer 373 sequencer and the following primers: pUC/M13 forward, pUC/M13 reverse, Seqsmt5 and Seqsmt3. The sequences of these primers are given in Table 1.

The following results were obtained

A pool of tobacco cDNAs were generated by the reverse transcription of a total RNA preparation primed by a oligonucleotide directed towards the poly-A tail, RoRidT. The cDNA pool was used as template to amplify the full length Ntsmt1-1 cDNA using gene specific primers, Ntsmt13 and Ntsmt15. A PCR product that corresponds to the expected size, 1040 bp, was amplified. This PCR product was ligated into pGEM-T Easy and insert containing clones were selected based on blue-white screening. Forty clones were screened by restriction digestion analysis and five clones containing an insert of the correct size (1040 bp) were chosen for sequencing.

Five insert containing clones, pGBW1, pGW6, pGW12, pGW16 and pGW18, were sequenced using primers pUC/M13 forward, pUC/M13 reverse, Seqsmt5 and Seqsmt3. The sequences obtained were aligned to the Ntsmt1-1 sequence published by Bouvier-Nave et al. (1998). This demonstrated that the amplified cDNA indeed was the full length Ntsmt1-1 cDNA (see FIG. 1B). However, as shown in Table 1B there were minor discrepancies in the sequenced clones as compared to the published clone.

TABLE 1B
Discrepancies between the sequenced clones and
the published Ntsmt1-1 cDNA

| Clone | Base pair substitution |
|---|---|
| pGBW1 | T551→G, G553→T |
| pGW6 | T551→G, G553→T |
| pGW12 | T551→G, G553→T |
| pGW16 | T551→G, G553→T, T789→C |
| pGW18 | T551→G, G553→T, A710→G |

It is difficult to assess whether the T551→G and G553→T base pair substitutions have arisen from infidelity during the reverse transcription or PCR amplification, or if the differences found are due to natural variation or sequencing errors. It is however clear that the codon change, TTG to GTT, leads to a very conserved amino acid substitution, namely leucine to valine. Valine and leucine both contain aliphatic side chain that only differ in length by one carbon atom. Hence even though this amino acid substitution may have been introduced by the cloning procedure it is highly unlikely that the functionality of the enzyme has been altered.

### EXAMPLE 2

### Construction of Plant Transformation Vectors

The Qiagen mini prep kit was used to obtain plasmid DNA for sequencing and sub-cloning procedures. The Qiagen PCR purification kit and gel extraction kit were used to purify DNA fragments obtained by PCR amplification or restriction enzyme digestion, respectively.

The Ntsmt1-1 gene in accordance to example 1 was amplified from vector pGBW1 by PCR using standard conditions. The primers used, clsmt5 and clsmt3, each contained a restriction enzyme site in order to facilitate cloning. A mix of Taq and Pfu (10%) DNA polymerase was used to ensure high amplification fidelity during the amplification. The amplification products were separated on an agarose gel (1.2%) and the fragment corresponding to the 1040 bp Ntsmt1-1 gene was excised and purified.

Sequencing was performed using an automatic Perkin Elmer 373 sequencer and fluorescently labelled nucleotides. Vectors pNH6 and pNH14 were sequenced using primers CERV1S (or ACP5), seqsmt3, seqsmt5, clsmt3, clsmt5 and NosAs. Vector pNH7 was sequenced using primers CERV1S and NosAs and pNH19 was sequenced using primers ACP5 and NosAs. The sequences of all primers used are listed in Table 1.

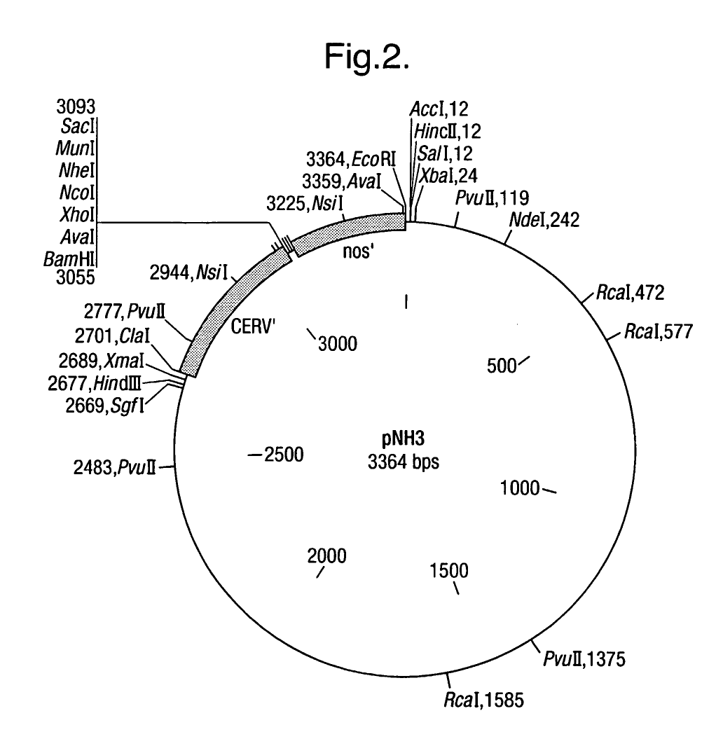

The Nicotiana tabaccum Ntsmt1-1 was amplified by PCR from plasmid pGBW1 using primers clsmt3 and clsmt5 as described above. This introduced restriction enzymes sites AflIII and EcoRI in the 5′ and 3′ terminus, respectively. The resulting amplification product was digested by AflIII and EcoRI and inserted into vector pNH3 (FIG. 2) between the

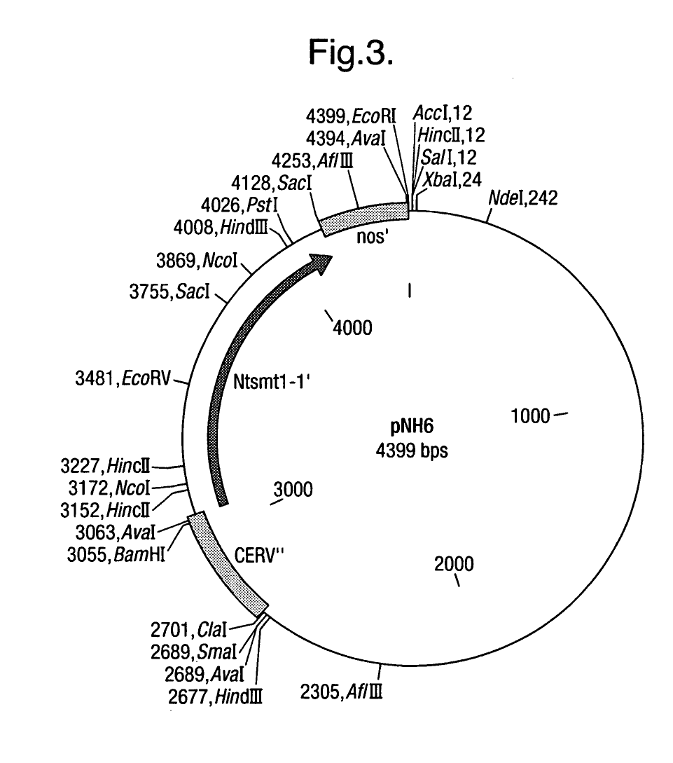

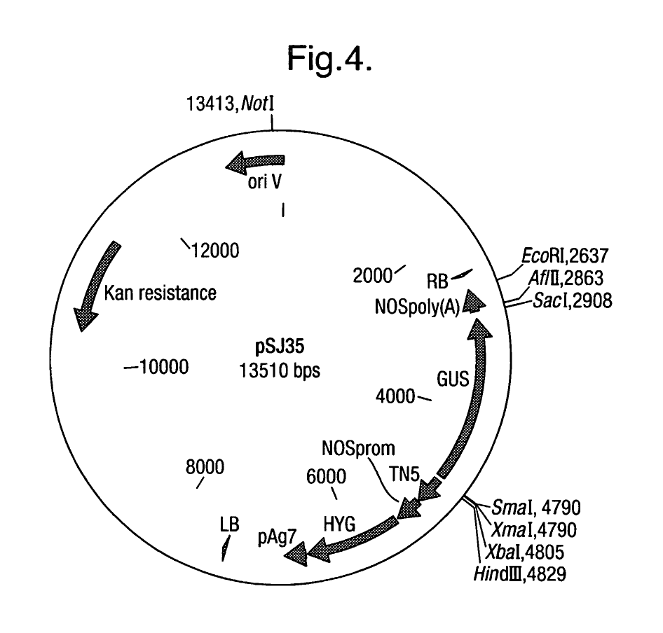

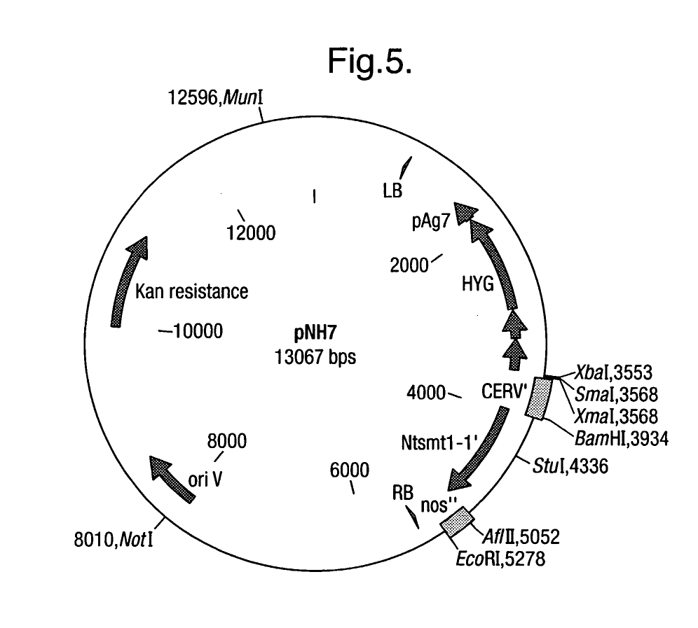

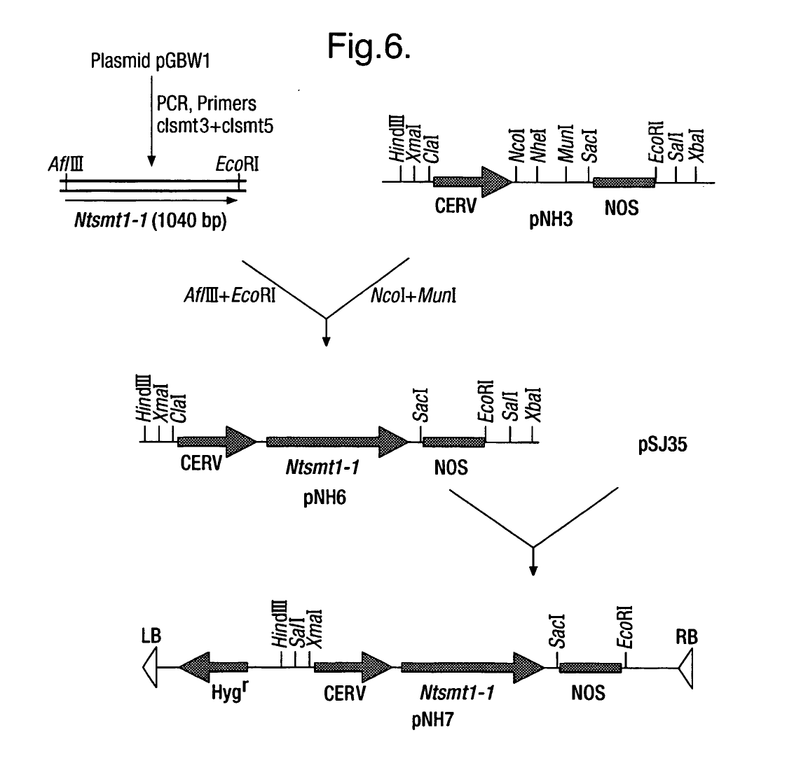

Carnation Etched Ring Virus (CERV) promoter and the Nopaline Synthase (NOS) terminator rendering vector pNH6 (FIG. 3). Vector pNH6 was digested by XmaI and EcoRI to excise the CERV-Ntsmt1-1-NOS cassette. This cassette was ligated into binary T-DNA vector pSJ35 (FIG. 4) resulting in vector pNH7 (FIG. 5). All cloning steps leading to vector pNH7 are summarised in FIG. 6. The entire Ntsmt1-1 gene in pNH6 was sequenced to confirm that it was amplified without introducing errors. Moreover, the linker regions between promoter, terminator and structural gene were also sequenced in vector pNH7 to confirm their integrity. The sequencing experiments demonstrated that the Ntsmt1-1 gene was amplified without errors and that the junctions between promoter, structural gene and terminator were also error free.

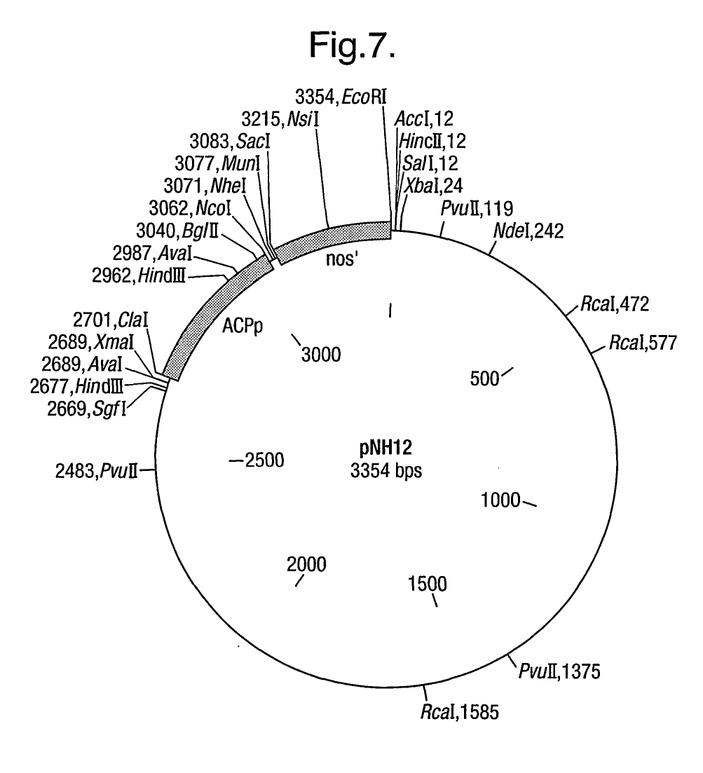

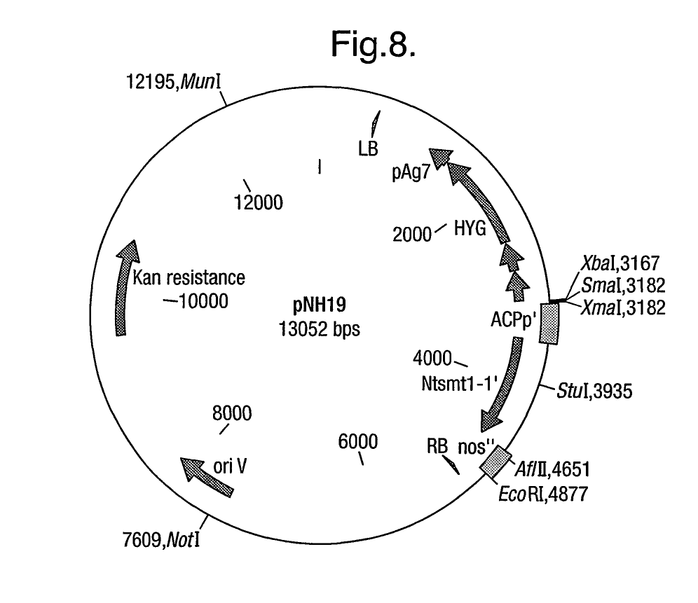

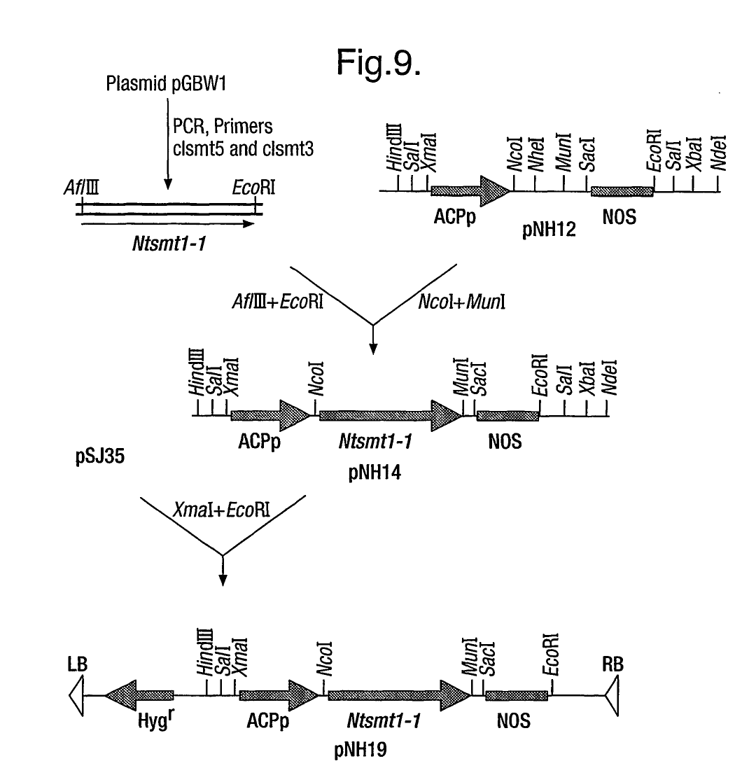

The Nicotiana tabaccum Ntsmt1-1 was amplified by PCR from plasmid pGBW1 using primers clsmt3 and clsmt5 as described above. This step introduced restriction enzymes in either end via the PCR primers (AflIII and EcoRI). The resulting amplification product was digested by AflIII and EcoRI and inserted into vector pNH12 (FIG. 7) between the NcoI and the MunI sites rendering vector pNH14. Vector pNH14 was digested by XmaI and EcoRI excising the ACPp-Ntsmt1-1-NOS (nopaline synthase terminator) cassette. This fragment was ligated into binary vector pSJ35 resulting in expression vector pNH19 (FIG. 8). All cloning steps leading to vector pNH19 are summarised in FIG. 9. Vector pNH19 was sequenced with primers ACP5, Ntsmt15 and NosAs. The sequencing results demonstrated that the junctions between the promoter and the structural gene as well as the terminator and the structural gene were error free.

### EXAMPLE 3

### Transformation of Tobacco

A. Binary vectors pNH7 (table 2 and 3) or pNH19 (table 4) 5 were transformed into Agrobacterium tumefaciens pGV3850 using triparental mating as described in Rogers et al 1988: Use of co-integrating Ti-plasmid vectors in Plant Molecular Biology Manual, eds Galvin & Schilperoort, Kluwer Academic Press. Transformants were analysed for presence of the gene of interest by PCR.

PCR positive cultures were used to inoculate a 10 ml Lennox media broth containing kanamycin 50 μg/ml and rifampicin 50 μg/ml. The overnight culture was spun down at 3000 g and resuspended in an equal volume of MS media (3% sucrose). Leaf segments were cut from young Nicotiana tabacum L. cv. SR1 leaves from plants grown in tissue culture. Segments were placed directly into the agrobacterium solution and left for 10 minutes. The segments were then removed and placed upper surface down on feeder plates (10 per plate) and left for 2 days in low light at 22° C. The leaf segments were then placed on tobacco shooting media with hormones containing cefotaxime 500 μg/ml and kanamycin 50 μg/ml with the upper surface up and placed in a growth room at 24° C. with a 16 hrs light 8 hrs dark regime. Three weeks later the callusing segments were transferred to tubs of tobacco shooting media. Once formed, shoots were excised and placed on tobacco shooting media without hormones containing cefotaxime 500 μg/ml and kanamycin 50 μg/ml to root. Rooted plants were then potted up into a 50% perlite/50% compost mixture and placed in a propagator. After 1 week the plants were removed from the propagator and subsequently potted up into 5 inch pots. Once flowering had begun, paper bags were placed over the flowers to prevent cross-pollination. When flowering had finished and pods formed, the bags were removed. Mature leaves and seed from dry pods were harvested and stored at −80° C. for subsequent sterol analysis. Immature leaves and 11-14 days after anthesis (daa) and 17 daa seeds were collected for enzyme analysis.

B. The plant tissues obtained in as above were assayed for SMT activity. All steps were carried out at 4° C. unless otherwise stated. Homogenisation buffer was 0.2M potassium phosphate, pH 7.5 containing 0.35M sorbitol, 10 mM EDTA, 5 mM MgCl_{2,} 5 mM glutathione and 4 g/100 ml insoluble polyvinylpolypyrrolidone (PVPP).

Freshly harvested tobacco leaves (2-4 cm length) were homogenised in the ratio 1:10 leaf: buffer (v/w) using an Ultra-turrax at maximum speed. After centrifugation for 5 min at 1,200 g, the resulting supernatant was further clarified by re-centrifugation at 1,200 g for 5 min. Maturing seeds (80-100 mg), stored at -80° C., were homogenised in the ratio 1:10 seed:buffer (w/v) using an Ultra-turrax at maximum speed (total homogenate). [17 days after anthesis (daa) seeds were harvested for the 35S construct transgenics and 11-14 daa seeds for the ACP construct transgenics]. Before extracting enzyme activity, 400 μl aliquots were centrifuged for 5 min at 1,200 g. The supernatant was removed with a syringe and discarded. The lipid and pellet fractions were extracted together with 400 μl buffer containing 10 mM CHAPS. The samples were whirlimixed and left on ice for 20 min. After centrifugation at 1,200 g for 5 min, the supernatant and floating lipid layer were retained. The pellet was re-extracted with 400 μl buffer containing 10 mM CHAPS. After a further 20 min on ice, the sample was centrifuged for 5 min at 1,200 g. The supernatant/lipid fractions were pooled and assayed immediately for enzyme activity.

The reaction mixture, assay conditions and TLC analysis were adapted from those described by Nes et al (1991) and Schaller et al (1998). The standard assay system consisted of 50 μM [¹⁴C-methyl] SAM (50 nCi), 125 μM lanosterol emulsified in Tween 80 and enzyme extract (80 μl) in a final volume of 100 μl. Final concentration of Tween 80 was 0.1% (v/v). Control assays had enzyme omitted. After incubation for 1 hour at 30° C., reactions were terminated by the addition of 100 μl 12% KOH in ethanol. Lanosterol and cholesterol, 15μg each, were added as carriers. The neutral lipids were extracted with hexane (2×600 μl) and the combined eluant was evaporated to dryness under nitrogen. The lipid residue was resuspended in 15 μl toluene. Sample (10 μl) was applied to pre-coated silica TLC plates (analytical, Merck) and chromatographed with dichloromethane. 4-desmethyl and 4,4-dimethyl sterols were visualised with iodine vapour. The bands corresponding to lanosterol (4,4-dimethyl sterol) were scraped off into scintillation vials. Liquid scintillation cocktail (Readysafe, Beckman) was added and radioactivity was monitored with a Beckman LS650 liquid scintillation counter.

C. For sterol analysis, the plant tissue obtained in as above is freeze-dried, then ground to a fine powder. 250 μl of 0.2% w/v dihydrocholesterol dissolved in chloroform is pipetted into a screw-top septum vial. After removal of solvent, an amount of the plant tissue (50 mg) is added to the vial, and total lipid extracted with 5 ml of a 2:1 v/v mixture of chloroform:methanol. The vial is capped and placed in a hot block maintained at 80-85° C. After 30 minutes the contents are filtered and the vial is washed 10 out with a second 5 ml aliquot of the chloroform:methanol mixture. The contents of the vial are filtered once more and the filtrates combined. The solvent portion of the filtrate is blown off using a stream of nitrogen gas to isolate the lipid residue.

The lipid fraction is then subjected to transmethylation by heating at 80-85° C. in 1 ml of toluene and 2 ml of 0.5N sodium methoxide in methanol. After 30 minutes, 2 ml of a 14% boron trifluoride solution in methanol is added and heated for a further 10 minutes at 80-85° C. After cooling, 2-3 ml of diethyl ether followed by 5 ml of deionised water are added. The ether fraction is removed and a further ether extraction carried out. The ether fractions are combined, backwashed with approx. 5 ml of water and dried overnight over anhydrous sodium sulphate. The ether phase is filtered and the solvent removed using a stream of nitrogen gas.

Sterols are dissolved in 300-400 μL of toluene and silylated by the addition of 200 μl of 95:5 N,O-bis(trimethylsilyl)acetamide:trimethylchlorosilane followed by incubation at 50° C. for 10 minutes. GC analysis is carried out using a 25 m×0.32 mm i.d. (0.25 μm film thickness) 5% BPX5 column (ex SGE) in a Perkin-Elmer 8420 GC. The temperature program is 180-240° C. at 10° C./min, followed by 240-355° C. at 15° C./min. and, finally, 5 min. at 355° C. The FID temperature is 380° C. and the helium pressure 10 psi. A volume of 1.0 μl is injected onto the column. A GC response factor of 1.0 for each of the sterols with respect to the dihydrocholesterol internal calibrant is assumed.

The five main sterol peaks (cholesterol, campesterol, stigmasterol, β-sitosterol, isofucosterol), the intermediate compound cycloartenol, and cholesterol were identified by comparison with authentic samples and library spectra following GC-MS analysis (Hewlett Packard 5890 Series 2 Plus GC interfaced to a 5972A mass selective detector) using a 30 m ×0.25 mm i.d. (0.25 μm film thickness) HP5-MS column. The oven temperature program was 100-320° C. at 10° C./min, then 8 min. at 320° C. Electron impact spectra were recorded at 70 eV and an electron multiplier voltage of 2494 V. A helium flow rate of 1 ml/min at constant flow and a 1.0 μl splitless injection were employed. The MS data range was 65-520 Daltons.

The reproducibility of this methodology was confirmed by repeated analysis of a particular batch of wild type tobacco seed. The amount of each sterol or cholesterol in plant tissue is expressed as a percentage of the dry sample weight.

### EXAMPLE 4

### Sterol Levels in Transformed Tobacco Tissues

Table 2 shows the sterol analysis of mature leaves obtained from tobacco transformed with Ntsmt1 cDNA under the control of the CERV promoter. Leaves from 10 independent transgenic plants (NH7) were analysed along with leaves from 3 independent untransformed plants (SR1) which had been generated via tissue culture. The total sterol content of the SR1 control leaves ranged from 0.228-0.310% dry weight. The Ntsmtl transgenic leaves contained total sterol contents of up to 0.335% dry weight. Table 2 also shows that cholesterol levels for the leaves of the SRI controls ranged from 0.0183 to 0.246% while for the NH7 plants levels down to 0.000% (or below detection) were observed.

These results show that although there is some increase in sterol levels of transgenic leaves the variation in sterol contents is quite high.

For cholesterol levels the effect is more pronounced, illustrating that surprisingly the level of cholesterol in leaves can be significantly lowered. This lowering seems to correlate to an increase in the SMTI activity.

TABLE 2
CERV-Ntsmt1 transformed tobacco leaf

| | | | | | | | | | SMT | | | | | | | |
|---|---|---|---|---|---|---|---|---|---|---|---|---|---|---|---|---|
| | | | | | | | | | activity | | | | | | | |
| | | | | | | | | | pmol/ | | | | | | | |
| Sample | Total sterols as % of dry wt | | | | | | | | mg | Sterol as % of Total Sterol | | | | | | |
| no. | chol | camp | stig | sito | isofuc | cycloart | 24mca | Total | prot/hr | chol | camp | stig | sito | isofuc | cycloart | 24mca |
| NH7 27 | 0.0000 | 0.1181 | 0.1244 | 0.0774 | 0.0109 | 0.0042 | 0.0000 | 0.335 | 45 | 0.0 | 35.3 | 37.1 | 23.1 | 3.3 | 1.2 | 0.0 |
| NH7 30 | 0.0098 | 0.1017 | 0.1309 | 0.0687 | 0.0097 | 0.0000 | 0.0000 | 0.321 | 44 | 3.1 | 31.7 | 40.8 | 21.4 | 3.0 | 0.0 | 0.0 |
| NH7 35 | 0.0095 | 0.0985 | 0.0862 | 0.0551 | 0.0107 | 0.0052 | 0.0000 | 0.265 | 54 | 3.6 | 37.1 | 32.5 | 20.8 | 4.0 | 2.0 | 0.0 |
| NH7 45 | 0.0023 | 0.0892 | 0.0779 | 0.0598 | 0.0108 | 0.0051 | 0.0000 | 0.245 | 61 | 0.9 | 36.4 | 31.8 | 24.4 | 4.4 | 2.1 | 0.0 |
| NH7 34 | 0.0043 | 0.0924 | 0.0776 | 0.0549 | 0.0105 | 0.0000 | 0.0000 | 0.240 | 41 | 1.8 | 38.6 | 32.4 | 22.9 | 4.4 | 0.0 | 0.0 |
| NH7 31 | 0.0048 | 0.0851 | 0.0845 | 0.0531 | 0.0110 | 0.0000 | 0.0000 | 0.238 | 41 | 2.0 | 35.7 | 35.4 | 22.3 | 4.6 | 0.0 | 0.0 |
| NH7 21 | 0.0244 | 0.0696 | 0.0772 | 0.0264 | 0.0223 | 0.0155 | 0.0000 | 0.235 | 6 | 10.4 | 29.6 | 32.8 | 11.2 | 9.5 | 6.6 | 0.0 |
| NH7 28 | 0.0049 | 0.0894 | 0.0838 | 0.0442 | 0.0079 | 0.0000 | 0.0000 | 0.230 | 49 | 2.1 | 38.8 | 36.4 | 19.2 | 3.4 | 0.0 | 0.0 |
| NH7 1 | 0.0067 | 0.0751 | 0.0671 | 0.0412 | 0.0115 | 0.0000 | 0.0000 | 0.202 | 5 | 3.3 | 37.3 | 33.3 | 20.4 | 5.7 | 0.0 | 0.0 |
| NH7 22 | 0.0032 | 0.0662 | 0.0600 | 0.0363 | 0.0120 | 0.0000 | 0.0000 | 0.178 | 48 | 1.8 | 37.2 | 33.8 | 20.4 | 6.8 | 0.0 | 0.0 |
| SR1 2 | 0.0246 | 0.0992 | 0.1164 | 0.0481 | 0.0151 | 0.0067 | 0.0000 | 0.310 | 4 | 7.9 | 32.0 | 37.5 | 15.5 | 4.9 | 2.2 | 0.0 |
| SR1 7 | 0.0184 | 0.0784 | 0.0886 | 0.0412 | 0.0224 | 0.0099 | 0.0000 | 0.259 | 15 | 7.1 | 30.3 | 34.2 | 15.9 | 8.7 | 3.8 | 0.0 |
| SR1 1 | 0.0183 | 0.0763 | 0.0809 | 0.0294 | 0.0172 | 0.0054 | 0.0000 | 0.228 | 16 | 8.0 | 33.5 | 35.6 | 12.9 | 7.5 | 2.4 | 0.0 |

Table 3 shows the sterol analysis of mature seeds obtained from tobacco transformed with Ntsmt1 cDNA under then control of the CERV promoter. Seeds of 29 independent transgenic plants (NH7) were analysed along with seeds from 9 independent untransformed plants (SR1) as above and 8 independent plants transformed with SJ35, as vector controls (pVEC). The total sterol content of the SR1 control seeds ranged from 0.279%-0.380% dry weight with a mean of 0.337% and that of the pVEC controls from 0.323%-0.376% with a mean of 0.353 %. The Ntsmtl transgenics seeds contained total sterol contents up to 0.451% dry weight, representing increases of up to 35%.

TABLE 3
CERV-Ntsmt1 transformed tobacco seed

| | | | | | | | | | SMT | | | | | | | |
|---|---|---|---|---|---|---|---|---|---|---|---|---|---|---|---|---|
| | | | | | | | | | activity | | | | | | | |
| | | | | | | | | | pmol/ | | | | | | | |
| Sample | Total sterols as % of dry wt | | | | | | | | mg | Sterol as % of Total Sterol | | | | | | |
| no. | chol | camp | stig | sito | isofuc | cycloart | 24mca | Total | prot/hr | chol | camp | stig | sito | isofuc | cycloart | 24mca |
| NH7 27 | 0.0229 | 0.0715 | 0.0397 | 0.1993 | 0.0979 | 0.0196 | 0.0000 | 0.451 | 22 | 5.1 | 15.9 | 8.8 | 44.2 | 21.7 | 4.3 | 0.0 |
| NH7 42 | 0.0238 | 0.0588 | 0.0381 | 0.1705 | 0.0838 | 0.0307 | 0.0000 | 0.406 | 21 | 5.9 | 14.5 | 9.4 | 42.0 | 20.7 | 7.6 | 0.0 |
| NH7 34 | 0.0211 | 0.0688 | 0.0370 | 0.1737 | 0.0859 | 0.0191 | 0.0000 | 0.406 | 21 | 5.2 | 17.0 | 9.1 | 42.8 | 21.2 | 4.7 | 0.0 |
| NH7 28 | 0.0210 | 0.0621 | 0.0408 | 0.1771 | 0.0852 | 0.0150 | 0.0000 | 0.401 | 52 | 5.2 | 15.5 | 10.2 | 44.2 | 21.2 | 3.7 | 0.0 |
| NH7 7 | 0.0207 | 0.0576 | 0.0354 | 0.1764 | 0.0821 | 0.0228 | 0.0000 | 0.395 | 16 | 5.2 | 14.6 | 8.9 | 44.7 | 20.8 | 5.8 | 0.0 |
| NH7 1 | 0.0231 | 0.0582 | 0.0350 | 0.1696 | 0.0814 | 0.0247 | 0.0000 | 0.392 | 24 | 5.9 | 14.9 | 8.9 | 43.3 | 20.8 | 6.3 | 0.0 |
| NH7 8 | 0.0274 | 0.0564 | 0.0352 | 0.1646 | 0.0805 | 0.0248 | 0.0000 | 0.389 | 22 | 7.1 | 14.5 | 9.1 | 42.3 | 20.7 | 6.4 | 0.0 |
| NH7 29 | 0.0235 | 0.0567 | 0.0389 | 0.1655 | 0.0744 | 0.0292 | 0.0000 | 0.388 | 24 | 6.1 | 14.6 | 10.0 | 42.6 | 19.2 | 7.5 | 0.0 |
| NH7 30 | 0.0216 | 0.0586 | 0.0446 | 0.1682 | 0.0785 | 0.0164 | 0.0000 | 0.388 | 27 | 5.6 | 15.1 | 11.5 | 43.4 | 20.2 | 4.2 | 0.0 |
| NH7 35 | 0.0203 | 0.0566 | 0.0368 | 0.1750 | 0.0795 | 0.0162 | 0.0000 | 0.384 | 16 | 5.3 | 14.7 | 9.6 | 45.5 | 20.7 | 4.2 | 0.0 |
| NH7 31 | 0.0215 | 0.0520 | 0.0369 | 0.1710 | 0.0796 | 0.0209 | 0.0000 | 0.382 | 25 | 5.6 | 13.6 | 9.7 | 44.8 | 20.8 | 5.5 | 0.0 |
| NH7 17 | 0.0296 | 0.0555 | 0.0343 | 0.1578 | 0.0845 | 0.0193 | 0.0000 | 0.381 | | 7.8 | 14.6 | 9.0 | 41.4 | 22.2 | 5.1 | 0.0 |
| NH7 36 | 0.0202 | 0.0567 | 0.0395 | 0.1685 | 0.0723 | 0.0203 | 0.0000 | 0.378 | 32 | 5.4 | 15.0 | 10.5 | 44.6 | 19.1 | 5.4 | 0.0 |
| NH7 40 | 0.0288 | 0.0512 | 0.0361 | 0.1418 | 0.0725 | 0.0398 | 0.0000 | 0.370 | | 7.8 | 13.8 | 9.8 | 38.3 | 19.6 | 10.7 | 0.0 |
| NH7 33 | 0.0180 | 0.0561 | 0.0343 | 0.1666 | 0.0750 | 0.0194 | 0.0000 | 0.369 | 65 | 4.9 | 15.2 | 9.3 | 45.1 | 20.3 | 5.3 | 0.0 |
| NH7 22 | 0.0166 | 0.0660 | 0.0402 | 0.1588 | 0.0773 | 0.0102 | 0.0000 | 0.369 | 37 | 4.5 | 17.9 | 10.9 | 43.0 | 21.0 | 2.8 | 0.0 |
| NH7 45 | 0.0144 | 0.0566 | 0.0395 | 0.1695 | 0.0645 | 0.0161 | 0.0000 | 0.361 | 48 | 4.0 | 15.7 | 11.0 | 47.0 | 17.9 | 4.5 | 0.0 |
| NH7 10 | 0.0212 | 0.0534 | 0.0381 | 0.1568 | 0.0646 | 0.0254 | 0.0000 | 0.360 | 9 | 5.9 | 14.9 | 10.6 | 43.6 | 18.0 | 7.1 | 0.0 |
| NH7 37 | 0.0291 | 0.0478 | 0.0330 | 0.1397 | 0.0644 | 0.0443 | 0.0000 | 0.358 | | 8.1 | 13.3 | 9.2 | 39.0 | 18.0 | 12.4 | 0.0 |
| NH7 44 | 0.0263 | 0.0471 | 0.0329 | 0.1395 | 0.0677 | 0.0427 | 0.0000 | 0.356 | 19 | 7.4 | 13.2 | 9.2 | 39.2 | 19.0 | 12.0 | 0.0 |
| NH7 38 | 0.0289 | 0.0468 | 0.0313 | 0.1361 | 0.0634 | 0.0481 | 0.0000 | 0.355 | 13 | 8.2 | 13.2 | 8.8 | 38.4 | 17.9 | 13.6 | 0.0 |
| NH7 13 | 0.0255 | 0.0473 | 0.0340 | 0.1435 | 0.0695 | 0.0345 | 0.0000 | 0.354 | 9 | 7.2 | 13.3 | 9.6 | 40.5 | 19.6 | 9.7 | 0.0 |
| NH7 32 | 0.0226 | 0.0443 | 0.0293 | 0.1479 | 0.0706 | 0.0387 | 0.0000 | 0.353 | | 6.4 | 12.5 | 8.3 | 41.9 | 20.0 | 10.9 | 0.0 |
| NH7 23 | 0.0251 | 0.0487 | 0.0338 | 0.1376 | 0.0693 | 0.0341 | 0.0000 | 0.349 | 13 | 7.2 | 14.0 | 9.7 | 39.5 | 19.9 | 9.8 | 0.0 |
| NH7 16 | 0.0224 | 0.0517 | 0.0408 | 0.1408 | 0.0599 | 0.0275 | 0.0000 | 0.343 | 20 | 6.5 | 15.1 | 11.9 | 41.0 | 17.5 | 8.0 | 0.0 |
| NH7 21 | 0.0298 | 0.0430 | 0.0342 | 0.1231 | 0.0539 | 0.0538 | 0.0000 | 0.338 | 9 | 8.8 | 12.7 | 10.1 | 36.4 | 15.9 | 15.9 | 0.0 |
| NH7 39 | 0.0241 | 0.0431 | 0.0336 | 0.1456 | 0.0617 | 0.0260 | 0.0000 | 0.334 | 22 | 7.2 | 12.9 | 10.1 | 43.6 | 18.5 | 7.8 | 0.0 |
| NH7 2 | 0.0267 | 0.0440 | 0.0351 | 0.1240 | 0.0484 | 0.0531 | 0.0000 | 0.331 | 5 | 8.1 | 13.3 | 10.6 | 37.4 | 14.6 | 16.0 | 0.0 |
| NH7 5 | 0.0328 | 0.0451 | 0.0353 | 0.1239 | 0.0583 | 0.0254 | 0.0000 | 0.321 | | 10.2 | 14.1 | 11.0 | 38.6 | 18.2 | 7.9 | 0.0 |
| SR1 8 | 0.0323 | 0.0532 | 0.0345 | 0.1528 | 0.0738 | 0.0338 | 0.0000 | 0.380 | 4 | 8.5 | 14.0 | 9.1 | 40.2 | 19.4 | 8.9 | 0.0 |
| SR1 1 | 0.0261 | 0.0512 | 0.0350 | 0.1480 | 0.0700 | 0.0484 | 0.0000 | 0.379 | | 6.9 | 13.5 | 9.2 | 39.1 | 18.5 | 12.8 | 0.0 |
| SR1 9 | 0.0267 | 0.0517 | 0.0400 | 0.1474 | 0.0718 | 0.0275 | 0.0000 | 0.365 | | 7.3 | 14.2 | 11.0 | 40.4 | 19.7 | 7.5 | 0.0 |
| SR1 2 | 0.0248 | 0.0500 | 0.0316 | 0.1478 | 0.0662 | 0.0383 | 0.0000 | 0.359 | | 6.9 | 13.9 | 8.8 | 41.2 | 18.5 | 10.7 | 0.0 |
| SR1 4 | 0.0237 | 0.0478 | 0.0369 | 0.1333 | 0.0607 | 0.0267 | 0.0000 | 0.329 | | 7.2 | 14.5 | 11.2 | 40.5 | 18.4 | 8.1 | 0.0 |
| SR1 10 | 0.0211 | 0.0489 | 0.0394 | 0.1356 | 0.0571 | 0.0237 | 0.0000 | 0.326 | 9 | 6.5 | 15.0 | 12.1 | 41.6 | 17.5 | 7.3 | 0.0 |
| SR1 7 | 0.0216 | 0.0444 | 0.0379 | 0.1377 | 0.0576 | 0.0156 | 0.0000 | 0.315 | 6 | 6.9 | 14.1 | 12.0 | 43.8 | 18.3 | 5.0 | 0.0 |
| SR1 6 | 0.0182 | 0.0449 | 0.0413 | 0.1332 | 0.0479 | 0.0202 | 0.0000 | 0.306 | 11 | 6.0 | 14.7 | 13.5 | 43.6 | 15.7 | 6.6 | 0.0 |
| SR1 3 | 0.0167 | 0.0466 | 0.0427 | 0.1176 | 0.0395 | 0.0155 | 0.0000 | 0.279 | 4 | 6.0 | 16.7 | 15.3 | 42.2 | 14.2 | 5.6 | 0.0 |
| pVEC 3 | 0.0279 | 0.0516 | 0.0373 | 0.1507 | 0.0768 | 0.0321 | 0.0000 | 0.376 | | 7.4 | 13.7 | 9.9 | 40.1 | 20.4 | 8.5 | 0.0 |
| pVEC 8 | 0.0276 | 0.0526 | 0.0361 | 0.1627 | 0.0766 | 0.0197 | 0.0000 | 0.375 | | 7.3 | 14.0 | 9.6 | 43.4 | 20.4 | 5.2 | 0.0 |
| pVEC 7 | 0.0241 | 0.0512 | 0.0361 | 0.1480 | 0.0687 | 0.0348 | 0.0000 | 0.363 | 5 | 6.6 | 14.1 | 9.9 | 40.8 | 18.9 | 9.6 | 0.0 |
| pVEC 4 | 0.0255 | 0.0495 | 0.0328 | 0.1480 | 0.0696 | 0.0340 | 0.0000 | 0.359 | 7 | 7.1 | 13.8 | 9.1 | 41.2 | 19.4 | 9.5 | 0.0 |
| pVEC 1 | 0.0250 | 0.0471 | 0.0333 | 0.1499 | 0.0717 | 0.0242 | 0.0000 | 0.351 | | 7.1 | 13.4 | 9.5 | 42.7 | 20.4 | 6.9 | 0.0 |
| pVEC 5 | 0.0280 | 0.0460 | 0.0311 | 0.1409 | 0.0684 | 0.0287 | 0.0000 | 0.343 | | 8.2 | 13.4 | 9.1 | 41.1 | 19.9 | 8.4 | 0.0 |
| pVEC | 0.0239 | 0.0472 | 0.0345 | 0.1401 | 0.0619 | 0.0211 | 0.0000 | 0.329 | 16 | 7.3 | 14.4 | 10.5 | 42.6 | 18.8 | 6.4 | 0.0 |
| 15 | | | | | | | | | | | | | | | | |
| pVEC 2 | 0.0187 | 0.0432 | 0.0379 | 0.1448 | 0.0536 | 0.0252 | 0.0000 | 0.323 | | 5.8 | 13.3 | 11.7 | 44.8 | 16.6 | 7.8 | 0.0 |

N.B. For total sterol data:-
SR1 average value = 0.337; standard deviation = 0.033
pVEC avge. value = 0.353; std. dev. = 0.018

Table 4 shows the sterol analysis of mature tobacco seeds obtained from tobacco transformed with Ntsmt1 cDNA under control of the ACP promoter. Again NH19 samples are in accordance to the invention and SR1 samples are control. The total sterol content of the SR1 control seeds ranged from 0.359%-0.398% dry weight with a mean of 0.376%. The Ntsmt1 transgenics seeds contained sterol contents up to 0.506% dry weight, representing increases of up to 35%. The average cholesterol content of the control seeds is 7.4%, but this is reduced to 3.2% in the highest sterol-containing transgenic seeds, representing a decrease of 53%.

TABLE 4
ACP-Ntsmt1 transformed tobacco seed

| | | | | | | | | | SMT | | | | | | | |
|---|---|---|---|---|---|---|---|---|---|---|---|---|---|---|---|---|
| | | | | | | | | | activity | | | | | | | |
| | | | | | | | | | pmol/ | | | | | | | |
| Sample | Total sterols as % of dry wt | | | | | | | | mg | Sterol as % of Total Sterol | | | | | | |
| no. | chol | camp | stig | sito | isofuc | cycloart | 24mca | Total | prot/hr | chol | camp | stig | sito | isofuc | cycloart | 24mca |
| NH19 | 0.0164 | 0.0891 | 0.0424 | 0.2243 | 0.1072 | 0.0266 | 0.0000 | 0.506 | 19.5 | 3.2 | 17.6 | 8.4 | 44.3 | 21.2 | 5.2 | 0.0 |
| 27 | | | | | | | | | | | | | | | | |
| NH19 | 0.0186 | 0.0709 | 0.0374 | 0.2011 | 0.0920 | 0.0259 | 0.0000 | 0.446 | 19.7 | 4.2 | 15.9 | 8.4 | 45.1 | 20.6 | 5.8 | 0.0 |
| 13 | | | | | | | | | | | | | | | | |
| NH19 | 0.0294 | 0.0636 | 0.0367 | 0.1879 | 0.0872 | 0.0335 | 0.0000 | 0.438 | 9.4 | 6.7 | 14.5 | 8.4 | 42.9 | 19.9 | 7.6 | 0.0 |
| 15 | | | | | | | | | | | | | | | | |
| NH19 | 0.0205 | 0.0661 | 0.0383 | 0.1933 | 0.0809 | 0.0309 | 0.0000 | 0.430 | 7.8 | 4.8 | 15.4 | 8.9 | 44.9 | 18.8 | 7.2 | 0.0 |
| 25 | | | | | | | | | | | | | | | | |
| NH19 | 0.0273 | 0.0656 | 0.0415 | 0.1696 | 0.0820 | 0.0342 | 0.0000 | 0.420 | 10.6 | 6.5 | 15.6 | 9.9 | 40.4 | 19.5 | 8.1 | 0.0 |
| 29 | | | | | | | | | | | | | | | | |
| NH19 | 0.0234 | 0.0658 | 0.0411 | 0.1706 | 0.0806 | 0.0314 | 0.0000 | 0.413 | 11.4 | 5.7 | 15.9 | 9.9 | 41.3 | 19.5 | 7.6 | 0.0 |
| 19 | | | | | | | | | | | | | | | | |
| NH19 | 0.0198 | 0.0638 | 0.0370 | 0.1783 | 0.0851 | 0.0263 | 0.0000 | 0.410 | 10.9 | 4.8 | 15.6 | 9.0 | 43.5 | 20.7 | 6.4 | 0.0 |
| 17 | | | | | | | | | | | | | | | | |
| NH19 | 0.0210 | 0.0644 | 0.0385 | 0.1778 | 0.0854 | 0.0222 | 0.0000 | 0.409 | 11.2 | 5.1 | 15.7 | 9.4 | 43.4 | 20.9 | 5.4 | 0.0 |
| 9 | | | | | | | | | | | | | | | | |
| NH19 | 0.0264 | 0.0604 | 0.0405 | 0.1694 | 0.0759 | 0.0355 | 0.0000 | 0.408 | 9.9 | 6.5 | 14.8 | 9.9 | 41.5 | 18.6 | 8.7 | 0.0 |
| 16 | | | | | | | | | | | | | | | | |
| NH19 | 0.0264 | 0.0625 | 0.0416 | 0.1491 | 0.0654 | 0.0618 | 0.0000 | 0.407 | 13.8 | 6.5 | 15.4 | 10.2 | 36.7 | 16.1 | 15.2 | 0.0 |
| 50 | | | | | | | | | | | | | | | | |
| NH19 | 0.0254 | 0.0617 | 0.0409 | 0.1550 | 0.0713 | 0.0464 | 0.0000 | 0.401 | 8.4 | 6.3 | 15.4 | 10.2 | 38.7 | 17.8 | 11.6 | 0.0 |
| 36 | | | | | | | | | | | | | | | | |
| NH19 | 0.0290 | 0.0563 | 0.0374 | 0.1577 | 0.0749 | 0.0450 | 0.0000 | 0.400 | | 7.2 | 14.1 | 9.4 | 39.4 | 18.7 | 11.2 | 0.0 |
| 28 | | | | | | | | | | | | | | | | |
| NH19 | 0.0259 | 0.0564 | 0.0377 | 0.1637 | 0.0738 | 0.0407 | 0.0000 | 0.398 | | 6.5 | 14.2 | 9.5 | 41.1 | 18.5 | 10.2 | 0.0 |
| 34 | | | | | | | | | | | | | | | | |
| NH19 | 0.0224 | 0.0571 | 0.0355 | 0.1725 | 0.0788 | 0.0285 | 0.0000 | 0.395 | 11.3 | 5.7 | 14.5 | 9.0 | 43.7 | 20.0 | 7.2 | 0.0 |
| 22 | | | | | | | | | | | | | | | | |
| NH19 | 0.0215 | 0.0605 | 0.0379 | 0.1714 | 0.0750 | 0.0280 | 0.0000 | 0.394 | 13.3 | 5.5 | 15.3 | 9.6 | 43.5 | 19.0 | 7.1 | 0.0 |
| 14 | | | | | | | | | | | | | | | | |
| NH19 | 0.0290 | 0.0587 | 0.0459 | 0.1402 | 0.0795 | 0.0391 | 0.0000 | 0.392 | 5.5 | 7.4 | 15.0 | 11.7 | 35.7 | 20.3 | 10.0 | 0.0 |
| 20 | | | | | | | | | | | | | | | | |
| NH19 | 0.0303 | 0.0504 | 0.0387 | 0.1443 | 0.0649 | 0.0619 | 0.0000 | 0.390 | 10.5 | 7.8 | 12.9 | 9.9 | 36.9 | 16.6 | 15.8 | 0.0 |
| 33 | | | | | | | | | | | | | | | | |
| NH19 | 0.0269 | 0.0509 | 0.0368 | 0.1455 | 0.0649 | 0.0603 | 0.0000 | 0.365 | | 7.0 | 13.2 | 9.6 | 37.8 | 16.9 | 15.6 | 0.0 |
| 11 | | | | | | | | | | | | | | | | |
| NH19 | 0.0234 | 0.0595 | 0.0449 | 0.1619 | 0.0654 | 0.0259 | 0.0000 | 0.381 | 11.5 | 6.1 | 15.6 | 11.8 | 42.5 | 17.2 | 6.8 | 0.0 |
| 32 | | | | | | | | | | | | | | | | |
| NH19 | 0.0256 | 0.0522 | 0.0353 | 0.1616 | 0.0708 | 0.0313 | 0.0000 | 0.377 | 7.4 | 6.8 | 13.8 | 9.4 | 42.9 | 18.8 | 8.3 | 0.0 |
| 5 | | | | | | | | | | | | | | | | |
| NH19 | 0.0242 | 0.0585 | 0.0384 | 0.1481 | 0.0686 | 0.0356 | 0.0000 | 0.373 | 3.2 | 6.5 | 15.7 | 10.3 | 39.7 | 18.4 | 9.5 | 0.0 |
| 10 | | | | | | | | | | | | | | | | |
| NH19 | 0.0335 | 0.0480 | 0.0359 | 0.1548 | 0.0724 | 0.0262 | 0.0000 | 0.371 | 6.8 | 9.0 | 12.9 | 9.7 | 41.8 | 19.5 | 7.1 | 0.0 |
| 6 | | | | | | | | | | | | | | | | |
| NH19 | 0.0269 | 0.0503 | 0.0368 | 0.1434 | 0.0611 | 0.0462 | 0.0000 | 0.365 | 6.2 | 7.4 | 13.8 | 10.1 | 39.3 | 16.7 | 12.7 | 0.0 |
| 7 | | | | | | | | | | | | | | | | |
| NH19 | 0.0308 | 0.0434 | 0.0336 | 0.1364 | 0.0642 | 0.0532 | 0.0000 | 0.362 | 8.4 | 8.5 | 12.0 | 9.3 | 37.7 | 17.8 | 14.7 | 0.0 |
| 24 | | | | | | | | | | | | | | | | |
| NH19 | 0.0330 | 0.0470 | 0.0338 | 0.1287 | 0.0586 | 0.0505 | 0.0000 | 0.352 | 5.8 | 9.4 | 13.4 | 9.6 | 36.6 | 16.7 | 14.4 | 0.0 |
| 8 | | | | | | | | | | | | | | | | |
| SR1 16 | 0.0271 | 0.0608 | 0.0432 | 0.1535 | 0.0779 | 0.0352 | 0.0000 | 0.398 | 8.9 | 6.8 | 15.3 | 10.9 | 38.6 | 19.6 | 8.9 | 0.0 |
| SR1 4 | 0.0298 | 0.0555 | 0.0418 | 0.1510 | 0.0800 | 0.0331 | 0.0000 | 0.391 | 1.2 | 7.6 | 14.2 | 10.7 | 38.6 | 20.4 | 8.5 | 0.0 |
| SR1 22 | 0.0351 | 0.0621 | 0.0448 | 0.1448 | 0.0722 | 0.0270 | 0.0000 | 0.386 | 9.9 | 9.1 | 16.1 | 11.6 | 37.5 | 18.7 | 7.0 | 0.0 |
| SR1 1 | 0.0265 | 0.0544 | 0.0383 | 0.1533 | 0.0760 | 0.0326 | 0.0000 | 0.381 | | 6.9 | 14.3 | 10.1 | 40.2 | 19.9 | 8.6 | 0.0 |
| SR1 14 | 0.0253 | 0.0580 | 0.0434 | 0.1515 | 0.0691 | 0.0308 | 0.0000 | 0.378 | 8.2 | 6.7 | 15.3 | 11.5 | 40.1 | 18.3 | 8.2 | 0.0 |
| SR1 24 | 0.0237 | 0.0625 | 0.0468 | 0.1451 | 0.0685 | 0.0252 | 0.0000 | 0.372 | | 6.4 | 16.8 | 12.6 | 39.0 | 18.4 | 6.8 | 0.0 |
| SR1 3 | 0.0273 | 0.0539 | 0.0356 | 0.1439 | 0.0767 | 0.0240 | 0.0000 | 0.361 | 5.8 | 7.5 | 14.9 | 9.9 | 39.8 | 21.2 | 6.6 | 0.0 |
| SR1 21 | 0.0274 | 0.0577 | 0.0436 | 0.1379 | 0.0714 | 0.0230 | 0.0000 | 0.361 | 6.2 | 7.6 | 16.0 | 12.1 | 38.2 | 19.8 | 6.4 | 0.0 |
| SR1 23 | 0.0291 | 0.0618 | 0.0464 | 0.1327 | 0.0662 | 0.0226 | 0.0000 | 0.359 | 7.7 | 8.1 | 17.2 | 12.9 | 37.0 | 18.4 | 6.3 | 0.0 |

SR1 average total sterol content = 0.376; standard deviation = 0.013

1. The use of a gene expressing a SMT1 to increase the level of sterols in the seeds of plants and/or decrease the level of cholesterol in plant tissue.

2. The use of according to claim 1, wherein the level of 4-desmethyl sterols is increased in the seeds of plants.

3. The use according to claim 1 wherein the level of one or more of the sterols selected from betasitosterol, sitostanol, stigmasterol, brassicasterol, campestanol, isofucosterol and campesterol is increased in the seeds of plants.

4. The use according to one or more of the preceding claims, wherein the level of sterols is increased in the seeds by at least 10%, more preferred at least 25%, most preferred at least 30%.

5. Use according to claim 1, wherein the plant tissue are seeds.

6. The use according to claim 1 or 5, wherein the seeds are oilseeds.

7. The use according to claim 6, wherein the oilseeds are from tobacco, canola, sunflower, rape or soy.

8. Method of obtaining seeds by
(a)Transforming a plant by:
1. transforming a plant cell with a recombinant DNA construct comprising a DNA segment encoding a polypeptide with SMT1 activity and a promoter for driving the expression of said polypeptide in said plant cell to form a transformed plant cell.
2. regenerating the transformed plant cell into the transgenic plant.
3. selecting transgenic plants that have enhanced levels of sterols or decreased levels of cholesterol in the seeds compared wild type strains of the same plant
(b)cultivating the transformed plant for one or more generations;
(c)harvesting seed from the plant grown under(b).

9. Seeds having enhanced level of sterols and produced by a plant having increased SMT1 activity.

10. Seeds according to claim 9 wherein the total level of sterols in the seeds is at least 0.400% of dry weight.

11. Method of obtaining oil comprising sterols by extracting oilseeds in accordance to claim 9.

12. Food product comprising an oil obtained in accordance to claim 11.

13. A method to increase the level of sterols in the seeds of plants and/or decrease the level of cholesterol in plant tissue by increasing the expression of SMT1 in said plants.

14. Plant tissue having decreased levels of cholesterol, said plant tissue being derived from a plant having increased SMT1 activity.

15. Plant tissue according to claim 14, wherein the cholesterol level is less than 0.01% on dry weight, more preferred less than 0.003%.


SEQUENCE LISTING (14 sequences)

SEQ ID NO: 1 - DNA, 60 bp, Artificial Sequence
  Description of Artificial SequencePrimer
aaggatccgtcgacatcgataatacgactcactatagggatttttttttttttttttttt

SEQ ID NO: 2 - DNA, 21 bp, Artificial Sequence
  Description of Artificial SequencePrimer
caacgccctccaaaaactgta

SEQ ID NO: 3 - DNA, 19 bp, Artificial Sequence
  Description of Artificial SequencePrimer
tgggaaagaaatgtcaaaa

SEQ ID NO: 4 - DNA, 20 bp, Artificial Sequence
  Description of Artificial SequencePrimer
tttcccagtcacgacgttgt

SEQ ID NO: 5 - DNA, 17 bp, Artificial Sequence
  Description of Artificial SequencePrimer
gtaaaacgacggccagt

SEQ ID NO: 6 - DNA, 26 bp, Artificial Sequence
  Description of Artificial SequencePrimer
ccatcgatctgattggtaagatatgg

SEQ ID NO: 7 - DNA, 23 bp, Artificial Sequence
  Description of Artificial SequencePrimer
ggaaggacatgtcaaaacaaggg

SEQ ID NO: 8 - DNA, 21 bp, Artificial Sequence
  Description of Artificial SequencePrimer
cggaattccatattactgaga

SEQ ID NO: 9 - DNA, 25 bp, Artificial Sequence
  Description of Artificial SequencePrimer
gtctgtctaaagtaaagtagatgcg

SEQ ID NO: 10 - DNA, 21 bp, Artificial Sequence
  Description of Artificial SequencePrimer
ccggcaacaggattcaatctt

SEQ ID NO: 11 - DNA, 19 bp, Artificial Sequence
  Description of Artificial SequencePrimer
agctactgagcgagaagtg

SEQ ID NO: 12 - DNA, 17 bp, Artificial Sequence
  Description of Artificial SequencePrimer
gcacccaggtggaaagg

SEQ ID NO: 13 - DNA, 1041 bp, Nicotiana tabacum
  CDS (1)..(1038)
atgtcaaaacaaggggcttttgatctggcatctggggttggtggcaaaattaacaaggaggaagttctctctgctgttgacaagtatgagaagtaccatggttattatggaggtgaagaagaagagagaaagaataactatactgacatggttaacaaatactatgatctttgcactagcttctacgaatacggctggggagagtcattccattttgcacccaggtggaaaggagaatcactccaagagagcattaaaaggcatgagcactttcttgccttgcaactgggattgaaaccaggacaaaaggtcttggacgtaggatgtggaattggtgggccgttaagagaaattgctcgattcagctctacatcagttacaggcctcaacaataatgaatatcagatatctaggggacaggtgttgaaccgcaaagtaggattggatcagacttgcaactttgtaaagggtgatttcatgaaaatgccattccctgacaatagctttgatgcagtgtacgcaatagaagctacctgccatgcaccagatccagttggatgctataaagagatttaccgggtgctgaagcctggtcaatgtttcgctgtgtatgagtggtgcatgaccgattcttacaaccccaataacgaagagcacaacaggatcaaggccgaaattgagctcggaaatggcctccctgaggttagattgacaacacagtgcctcgaagcagccaaacaagctggttttgaagttgtatgggacaaggatctggctgatgactcacctgttccatggtacttgcctttggatacgagtcacttctcgctcagtagcttccgcctaacagcagttggcagacttttcaccagaaatctggtttcggcgcttgaatacgtgggacttgctcctaaaggtagtcaaagggttcaagctttcttagagaaagctgcagaaggtcttgtcggtggtgccaagaaagggattttcacaccaatgtacttcttcgtggttcgcaagcccatttcagactctcagtaa

SEQ ID NO: 14 - protein, 346 aa, Nicotiana tabacum
MSKQGAFDLASGVGGKINKEEVLSAVDKYEKYHGYYGGEEEERKNNYTDMVNKYYDLCTSFYEYGWGESFHFAPRWKGESLQESIKRHEHFLALQLGLKPGQKVLDVGCGIGGPLREIARFSSTSVTGLNNNEYQISRGQVLNRKVGLDQTCNFVKGDFMKMPFPDNSFDAVYAIEATCHAPDPVGCYKEIYRVLKPGQCFAVYEWCMTDSYNPNNEEHNRIKAEIELGNGLPEVRLTTQCLEAAKQAGFEVVWDKDLADDSPVPWYLPLDTSHFSLSSFRLTAVGRLFTRNLVSALEYVGLAPKGSQRVQAFLEKAAEGLVGGAKKGIFTPMYFFVVRKPISDSQ